(19) United States
(12) Patent Application Publication
(10) Pub. No.: US 2024/0140977 A1
(43) Pub. Date: May 2, 2024

(54) REVERSIBLE TERMINATORS

(71) Applicant: Ansa Biotechnologies, Inc., Berkeley, CA (US)

(72) Inventors: Daniel Arlow, San Francisco, CA (US); Sebastian Palluk, Oakland, CA (US); Weidong Wu, Paris (FR)

(21) Appl. No.: 18/547,207

(22) PCT Filed: Feb. 25, 2022
(86) PCT No.: PCT/US2022/018020

§ 371 Date: Aug. 21, 2023

Related U.S. Application Data
(60) Provisional application No. 63/153,825, filed on Feb. 25, 2021.

(51) Int. Cl. C07H 19/10; C07H 19/14; C07H 19/20; C12Q 1/6869

(52) U.S. Cl. CPC C07H 19/10; C07H 19/14; C07H 19/20; C12Q 1/6869

(57) ABSTRACT

The present application is directed to, in part, nucleotide analogs with a novel design of a 3′-OH reversible terminator that comprises an enzyme-cleavable linkage.

### RELATED APPLICATIONS

This application claims the benefit of U.S. Provisional Patent Application No. 63/153,825 filed on Feb. 25, 2021, the contents of which are incorporated by reference in their entirety.

### BACKGROUND

The great majority of Next Generation Sequencing (NGS) performed today is based on “sequencing by synthesis” (SBS), in which the sequence of a primed template molecule is determined by a signal resulting from stepwise incorporation of complementary nucleotides by a polymerase (Goodwin et al., Nat Rev Genet. 2016 May 17; 17(6):333-51). Currently, the most popular method for SBS employs fluorescent “reversible terminator” nucleotides—nucleotides that are chemically modified to block elongation by a polymerase once they are incorporated into a primer. Once the identity of the incorporated cognate nucleotide is identified, the fluorescent reporter and terminating group are removed from the incorporated nucleotide, so that the terminating nucleotide is ready for subsequent extension by a polymerase. By repeating this cycle of template-dependent extension, detection, and deprotection, the sequence of the template molecule is identified from the sequence of fluorescence signals.

High throughput performance of such sequencing methods are needed to obtain fast, inexpensive, and accurate genome information. Such information is clinically important to facilitate personalized medicine based on genomic information for an individual, and is also desired to accurately correlate genomic sequences and mutations to specific diseases and conditions. However, current methodologies using reversible terminator sequencing are limited by short read length, which can be caused by inefficient cleavage or residual scars remaining on the synthesized nucleotide after cleavage of the terminating group and fluorescent reporter. What is needed, therefore, are novel modified nucleotides for sequencing by synthesis.

Standard de novo DNA synthesis performed today is based on the nucleoside phosphoramidite method, (generally referred to as “chemical synthesis”,) in which a desired sequence is synthesized by stepwise coupling of blocked monomers. The reactions are performed in organic solvents using highly reactive activated monomers, and the conditions cause side reactions that damage the growing chain, limiting the yield of full-length product. The impurities produced can be difficult or impractical to separate from the desired oligonucleotide product, limiting the usefulness of the method for producting sequences longer than approximately 200 bases.

Emerging fields such as Synthetic Biology would greatly benefit from direct synthesis of longer, higher-purity oligonucleotides that have been synthesized de novo. What is needed therefore, are improved methods of olignucleotide synthesis.

### SUMMARY

The present disclosure provides chemical compounds including reversible terminator molecules, i.e. nucleoside and nucleotide analogs which comprise a cleavable chemical group (the terminator group) covalently attached to the 3′ hydroxyl of the nucleotide sugar moiety. In addition, the reversible terminator molecules may comprise a detectable label attached to the base of the nucleotide through a cleavable linker. The terminator group and cleavable linker comprises a carbamate linkage and can be cleaved by an enzyme, such as an esterase or a carbamatase. The nucleotide analogs may be ribonucleotide or deoxyribonucleotide molecules and analogs, and derivatives thereof. Presence of the terminator group is designed to impede progress of polymerase enzymes used in methods of enzyme-based polynucleotide synthesis.

The present disclosure provides, in part, novel nucleotide analogs, where the 3′-OH group of the sugar is capped with a reversible chemical group blocking to temporarily terminate the polymerase reaction.

In one aspect, provided herein is a nucleotide analog of formula (I-A):

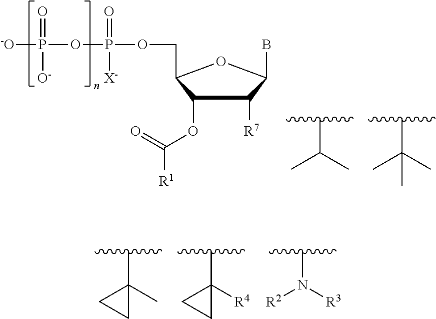

In another aspect, the present disclosure provides a nucleotide analog of formula (I-B):

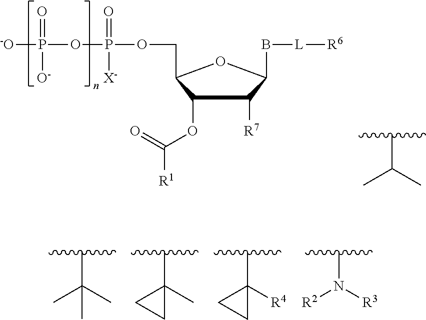

In another aspect, provided herein is a method of sequencing a single-stranded polynucleotide, comprising a) incorporating the nucleotide analog provided herein into a primer hybridized to said single-stranded polynucleotide using a polymerase; b) detecting the identity of said nucleotide analog; and c) contacting said nucleic acid molecule with an esterase or a carbamatase.

In another aspect, provided herein is a method of labeling a nucleic acid molecule, comprising a) incorporating the nucleotide analog provided herein into the nucleic acid molecule, wherein said nucleotide analog comprises or is bound to a label; and b) contacting the nucleic acid molecule with an esterase or a carbamatase.

In another aspect, provided herein is a method of synthesizing a single-stranded polynucleotide, comprising binding the nucleotide analog provided herein to the 3′ hydroxyl end of a polynucleotide using a polymerase. In some embodiments, to facilitate further synthesis, the method further comprises contacting the nucleotide analog incorporated into the single-stranded polynucleotide with an esterase or a carbamatase, thereby exposing the 3′ hydroxyl group of the added nucleotide analog.

### BRIEF DESCRIPTION OF THE DRAWINGS

FIG. 1 is a series of electropherograms showing characterization of the products from an oligonucleotide extension reaction using illustrative nucleotide analogs described herein. The vertical dashed line denotes the starter oligonucleotide peak. The arrow points to peaks containing extension products having the nucleotide analogs described herein.

### DETAILED DESCRIPTION

### Polynucleotide Synthesis

Highly accurate and efficient methods of polynucleotide synthesis in a controlled stepwise manner, are desired. Such a controlled synthesis reaction is useful for both de novo polynucleotide synthesis, e.g., to synthetically generate polynucleotide strands of specific desired sequence and determinable length, and template-directed polynucleotide synthesis, e.g., to determine a sequence of the complementary strand.

DNA-sequencing methods that are accurate and high throughput are essential for the detection and exploration of genomic information in an efficient and cost-effective manner. One such sequencing method that has shown significant promise is Sequencing by Synthesis (SBS). In SBS, a primer is hybridized to its target sequence and extended by nucleotide incorporation into the growing DNA strand using a DNA polymerase. A blocking group on the nucleotide prevents further nucleotide incorporation, and a reporter signal, such as a fluorophore, bound to the nucleotide, allows detection of the identity of the newly incorporated nucleotide. After detection of the identity of the incorporated nucleotide, the reporter signal is removed to ensure that the residual signal from the previous nucleotide incorporation does not affect the identification of the next incorporated nucleotide. In addition, the blocking group (i.e., reversible terminator) is removed to allow incorporation of the next incoming nucleotide complementary to the next base of the target sequence.

The process for using reversible terminator molecules in the context of SBS, SBE and like methodologies generally involves incorporation of a labeled nucleotide analog into the growing polynucleotide chain, followed by detection of the label, then cleavage of the nucleotide analog to remove the covalent modification blocking continued synthesis. The cleaving step may be accomplished using enzymatic cleavage.

Several techniques are available to achieve high-throughput sequencing. (See, Ansorge; Metzker; and Pareek et al., “Sequencing technologies and genome sequencing,” J Appl. Genet., 52(4):413-435, 2011, and references cited therein). In SBS, nucleotides are incorporated by a polymerase enzyme and because the nucleotides are differently labeled, the signal of the incorporated nucleotide, and therefore the identity of the nucleotide being incorporated into the growing synthetic polynucleotide strand, are determined by sensitive instruments, such as cameras.

In addition to sequencing, de novo DNA synthesis can also benefit from improved modified nucleotides with cleavable blocking groups. Methods for oligonucleotide synthesis using enzymes can be used to overcome the limitations of chemical DNA synthesis by performing reactions in mild, aqueous conditions using enzymes. In particular, in some embodiments, de novo polynucleotide synthesis is performed using a template-independent polymerase for stepwise addition of nucleoside triphosphates to a primer. A key challenge of this approach is to ensure that only a single nucleotide is added during each cycle, analogous to SBS. However, the blocking groups of existing reversible terminator nucleotides are removed chemically, and the conditions may result in side-reactions that result in undesireable damage to the synthesized polynucleotide, while also limiting the yield of full-length product. Provided herein are novel modified nucleotides useful for de novo polynucleotid synthesis.

In some embodiments, nucleotides modified to have a removal blocking group at the 3′-OH position can facilitate stepwise addition of nucleotides to a growing polynucleotide strand (e.g., for sequencing or de novo polynucleotide synthesis). This blocking group is bound to the nucleotide via a cleavable linkage, so that the blocking group can be removed to allow for a subsequent nucleotide addition to the 3′ end of a growing polynucleotide. Accordingly, the synthesis of labeled nucleotides with removable caps at its 3′-OH position is of interest to developing new SBS and de novo polynucleotide synthesis technologies.

Some SBS methods may use dye-labelled, modified nucleotides. These modified nucleotides may be incorporated specifically by an incorporating enzyme (e.g., a DNA polymerase), cleaved during or following fluorescence imaging, and extended as modified or natural bases in the growing strand in the ensuing cycles.

In the design of a labeled or unlabeled reversible chain terminator for stepwise controlled polynucleotide synthesis, is is desirable for the linker used as a chemically cleavable moiety for the reversible terminator blocking group or the cleavable moiety bound to the detectable label to have the following properties:
stability of the linker during the polymerase-mediated extension step,
the structure (geometry and size) and location of the linker must not prevent the recognition of the resulting labeled nucleotide by the DNA polymerase used for synthesis,
the linker is cleavable under mild conditions compatible with the stability of DNA biopolymers (single and/or double stranded), and
the incorporated nucleotide after cleavage of the linkers must not significantly interfere with polymerase activity for the next incoming nucleotide.

Sequencing and de novo polynucleotide synthesis using the presently disclosed reversible terminator molecules may be performed by any means available. For sequencing, generally, the categories of available technologies include, but are not limited to, sequencing-by-synthesis (SBS), sequencing by single-base-extension (SBE), sequencing-by-ligation, single molecule sequencing, and pyrosequencing, etc.

### Reversible Terminators

The present application is directed to, in part, a new class of nucleotide analogs (reversible terminators) with a novel design of a 3′ reversible terminator and/or a novel design of a linker bound to a detectable label. In some embodiments, these linkers are cleavable by an enzymatic method, such as by an esterase or a carbamatase enzyme.

In some embodiments, the reversible terminators as disclosed herein incorporate an ester or carbamate linkage between the oxygen atom of 3′-OH of the sugar and a moiety that is cleavable by an enzymatic method (e.g., as shown in Scheme 1). The reversible terminators of the present application are advantageous compared to the existing ones because they are stable under polymerase reaction conditions but can be easily cleaved in the presence of the appropriate enzyme. The enzyme may either cleave the ester or carbamate directly to release the 3′-OH (e.g as shown below), or, particularly in the case of carbamates, may cleave to a 3′ carbonate that will convert to the free 3′-OH upon spontaneous release of carbon dioxide.

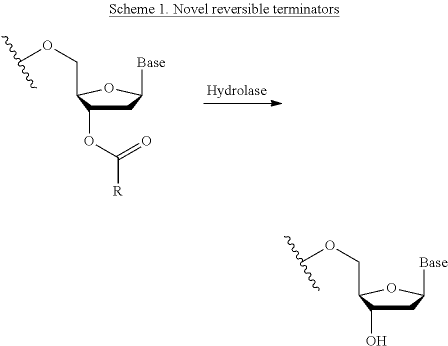

The present application is also directed to, in part, cleavable linkers to connect a detectable label to a nucleotide. The novel linkers as disclosed herein are advantageous compared to the existing linkers because they are stable under ambient conditions but can be efficiently cleaved in the presence of an esterase or a carbamatase. For example, a known linker may include an ester moiety, which may be cleavable by an esterase, as shown below.

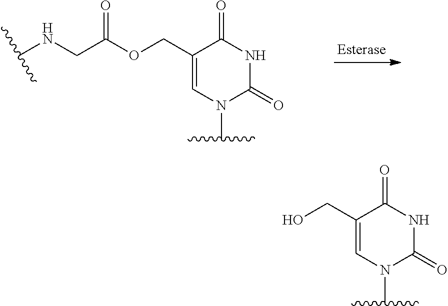 [SMILES: *NCC(=O)OCc1cn(*)c(=O)[nH]c1=O ; *n1cc(CO)c(=O)[nH]c1=O]

The hydroxymethyl moiety is the “scar” left on the base after the cleavage of the linker. After experimenting with several scar types that affect the DNA properties, the hydroxymethyl scar shown above was found to have favorable properties compared to larger scars. However, there are a challenge with this linker is that the ester is not as stable as needed during synthesis reactions.

After further investigations, the inventors have discovered that adding certain substitutents adjancent to the ester or alternatively by using a carbamate linkage can solve this stability problem.

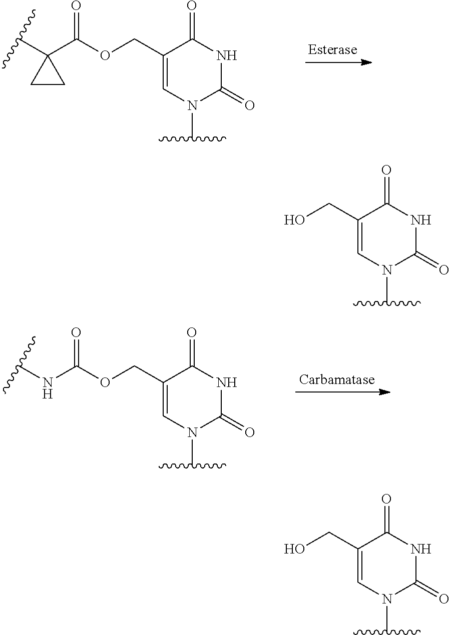 [SMILES: *n1cc(COC(=O)C2(*)CC2)c(=O)[nH]c1=O ; *n1cc(CO)c(=O)[nH]c1=O ; *NC(=O)OCc1cn(*)c(=O)[nH]c1=O ; *n1cc(CO)c(=O)[nH]c1=O]

The substituents adjacent to the ester stabilize the bond against spontaneous hydrolysis. Certain carbamates are also sufficiently hydrolytically stable. The linkers can be cleaved by an esterase or carbamatase.

Accordingly, the present disclosure provides, in part, reversible terminators (i.e. nucleotide analogs) bound to a nucleotide, where the detectable label and the nucleotide are covalently linked via a novel linker that comprises a stabilized ester or carbamate.

Such compositions are useful in novel methods of nucleotide synthesis, including for sequencing reactions, such as sequencing by synthesis.

The details of various embodiments of the nucleotide analogs and methods of use are set forth in the description below. Other features, objects, and advantages of the nucleotide analogs and methods of use will be apparent from the description and the drawings, and from the claims.

### Novel Nucleotide Analogs

Provided herein is a nucleotide analog of formula (I-A):

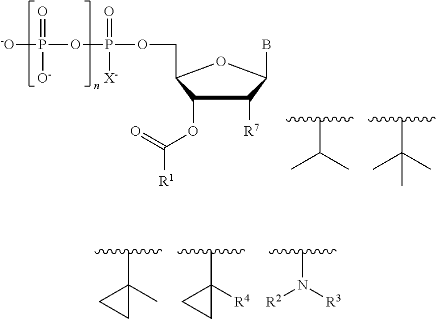

Also provided herein is a nucleotide analog of formula (I-B):

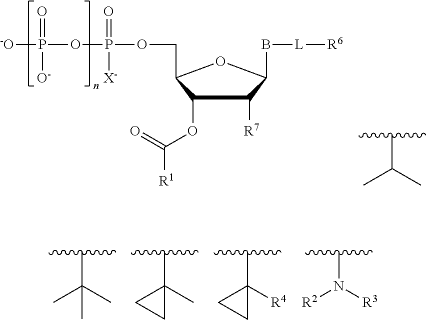

In some embodiments, B is a nucleotide base. In some embodiments, the base is a purine or a pyrimidine.

In some embodiments, Formula I-A or Formula I-B represents a nucleotide, such as a deoxyribonucleotide or a ribonucleotide.

The nucleotides described herein may contain adenine, cytosine, guanine, and thymine bases, and/or bases that base pair with a complementary nucleotide and are capable of being used as a template by a DNA or RNA polymerase, e.g., 7-deaza-7-propargylamino-adenine, 5-propargylamino-cytosine, 7-deaza-7-propargylamino-guanosine, 5-propargylamino-uridine, 7-deaza-7-hydroxymethyl-adenine, 5-hydroxymethyl-cytosine, 7-deaza-7-hydroxymethyl-guanosine, 5-hydroxymethyl-uridine, 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although others are known.

An exemplary set of nucleotides for synthesizing and/or sequencing a DNA molecule may include a modified deoxyribonucleotide triphosphate selected from deoxyriboadenosine triphosphate (dATP), deoxyriboguanosine triphosphate (dGTP), deoxyribocytidine triphosphate (dCTP), deoxyribothymidine triphosphate (dTTP), and/or other deoxyribonucleotides that base pair in the same way as those deoxyribonucleotides.

An exemplary set of nucleotides for synthesizing and/or sequencing an RNA molecule may include a modified ribonucleotide triphosphate selected from adenosine triphosphate (ATP), guanosine triphosphate (GTP), cytidine triphosphate (CTP), and uridine triphosphate (UTP), and/or other ribonucleotides that base pair in the same way as those ribonucleotide triphosphates.

In some embodiments, B is a scarred nucleotide base. The scarred nucleotide bases are the nucleotide bases that are substituted with a chemical moiety which is a portion of a linker (L). The scarred nucleotide bases are generated upon cleavage of L at the cleavable linkage.

In certain embodiments, the scarred nucleotide base is a nucleotide base substituted with —CH₂—OH, —C—C—CH₂—OH, or —C—C—CH₂—NHC(O)CH₂—OH.

In some embodiments, the scarred nucleotide base is selected from

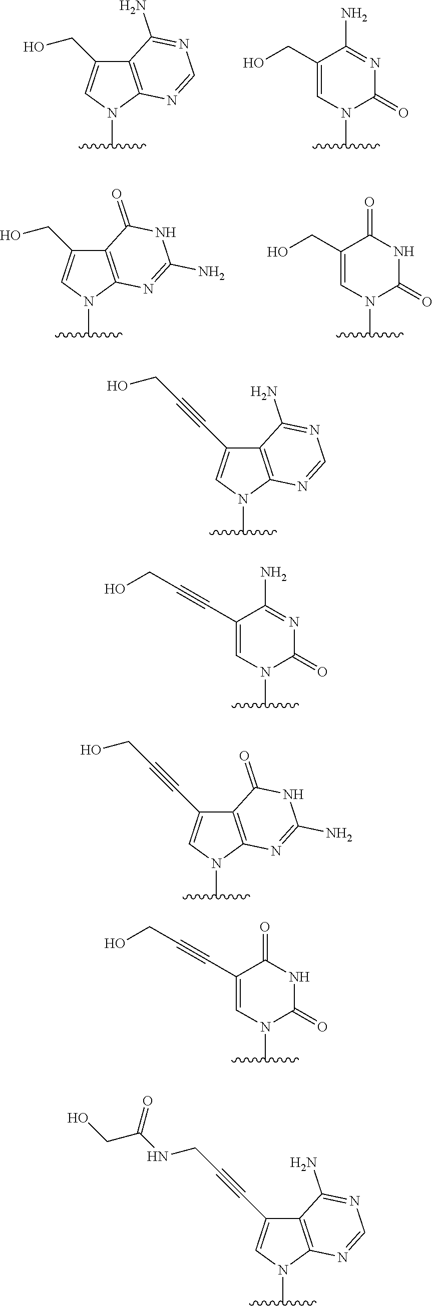

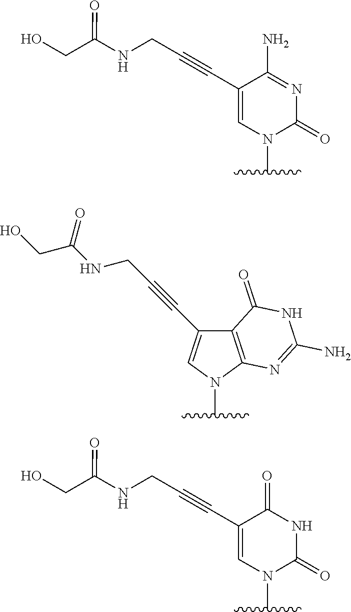 [SMILES: *n1cc(C#CCNC(=O)CO)c(N)nc1=O ; *n1cc(C#CCNC(=O)CO)c2c(=O)[nH]c(N)nc21 ; *n1cc(C#CCNC(=O)CO)c(=O)[nH]c1=O]

In other embodiments, X is O.

In certain embodiments, n is 0, 1, 2, 3, 4, or 5.

Linkers or contemplated herein are of sufficient length and stability to allow efficient hydrolysis or removal by chemical or enzymatic means. One end of the linker may be capable of being bound to or modified by a label group, such as a detectable label. The number of atoms in a linker, optionally derivatized by other functional groups, must be of sufficient length to allow either chemical or enzymatic cleavage of the blocking group, if the linker is attached to a blocking group or if the linker is attached to the detectable label.

While precise distances or separation may be varied for different reaction systems to obtain optimal results, in some cases, a linkage that maintains the bulky label moiety at some distance away from the nucleotide may be provided, e.g., a linker of 1 to 20 nm in length, to reduce steric crowding in enzyme binding sites. Therefore, the length of the linker may be, for example, 1-50 atoms in length, or 1-40 atoms in length, or 2-35 atoms in length, or 3 to 30 atoms in length, or 5 to 25 atoms in length, or 10 to 20 atoms in length.

Linkers may be comprised of any number of basic chemical starting blocks. For example, linkers may comprise linear or branched alkyl, alkenyl, or alkynyl chains, or combinations thereof, that provide a useful distance between the nucleobase and the detectable label. For instance, amino-alkyl linkers, e.g., amino-hexyl linkers, have been used to provide label attachment to nucleotide analogs. The longest chain of such linkers may include as many as 2 atoms, 3 atoms, 4 atoms, 5 atoms, 6 atoms, 7 atoms, 8 atoms, 9 atoms, 10 atoms, or even 11-35 atoms, or even 35-50 atoms. The linear or branched linker may also contain heteroatoms other than carbon, including, but not limited to, oxygen, sulfur, phosphate, and nitrogen. A polyoxyethylene chain (also commonly referred to as polyethyleneglycol, or PEG) is a preferred linker constituent due to the hydrophilic properties associated with polyoxyethylene. Insertion of heteroatom such as nitrogen and oxygen into the linkers may affect the solubility and stability of the linkers.

In preferred embodiments, linkers attaching the detectable label to the nucleotide comprise an aminomethyl or carboxymethyl spacer. In some embodiments, the linker comprises a carbonate, a carbamate, or a urea.

### Enzymatic Cleavage

In some embodiments, a linker comprising an ester is cleaved via an esterase. In the embodiments shown below, a linker is cleaved via esterase and the remaining nucleobase comprises a hydroxymethyl scar.

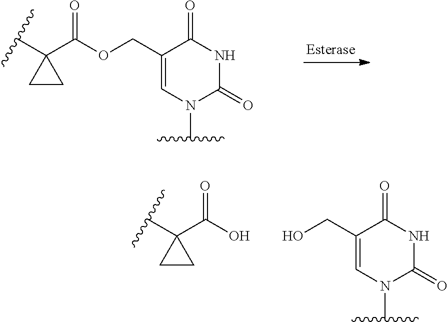 [SMILES: *n1cc(COC(=O)C2(*)CC2)c(=O)[nH]c1=O ; *C1(C(=O)O)CC1 ; *n1cc(CO)c(=O)[nH]c1=O]

In some embodiments, the esterase is classified under EC 3.1.1. In other embodiments, the esterase is a porcine liver esterase. In certain embodiments, the esterase is from rabbit liver, Rhizopus oryzae, Bacillus stearothermophilus, Bacillus subtilis, Saccharomyces cerevisiae, Methylobacterium populi, Paenibacillus barcinonensis, Pelobacter propionicus, or Pseudomonas fluorescens. In some embodiments, the esterase is also an amidase. In other embodiments, the esterase is selected from the group consisting of: Proteinase K, Subtilisin, and Chymotrypsin. In certain embodiments, the amidase is classified under EC 3.4 or EC 3.5.1.

In some embodiments, a linker comprising a carbamate is cleaved via a carbamatase. In the embodiments shown below, the linker is cleaved via carbamatase, and the hydrolysis intermediate spontaneously eliminates carbon dioxide to yield an alcohol. The resulting nucleobase comprises a hydroxymethyl scar.

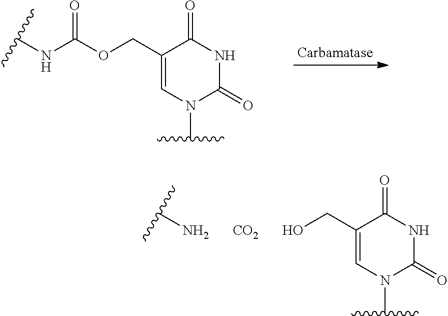

The linker comprising a carbamate can be cleaved with any suitable carbamatase. In some embodiments, the carbamatase is selected from the group consisting of: Urethanase, N_{α}-Benzyloxycarbonyl Amino Acid Urethane Hydrolase, Streptopain, Proteinase K, Subilisin, Chymotrypsin, Aminopeptidase, Trypsin, Papain, Bromelain, and Ficin. In other embodiments, the carbamatase is from Pyrococcus furiosus, Aspergillus oryzae, Aspergillus saitoi, Bacillus amyloliquefaciens, Bacillus licheniformis, Bacillus licheniformis, Bacillus licheniformis, Bacillus sp., Streptomyces griseus, Streptomyces griseus, and Streptomyces sp., Soil bacterium F-96, Enterococcus faecalis, Citrobacter sp., Micrococcus sp., Paenibacillus thiaminolyticus, Providencia rettgeri, Purpureocillium lilacinum, Rattus norvegicus, Rhodococcus hoagii, Rhodococcus hoagii TB-60, Rhodotorula mucilaginosa, Talaromyces variabilis, Spirastrella sp., Candida parapsilosis, or human. In some embodiments, the carbamatase is a variant of a wild-type, or naturally occurring carbamatase. In some embodiments, the carbamatase is an engineered carbamatase. In certain embodiments, the amidase is classified under EC 3.4 or EC 3.5.1. In some embodiments, any kind of enzyme with a hydrolase activity can be used to cleave the carbamate linkage, even if the enzyme has other activity. Illustrative carbamatase enzymes and uses thereof can be found in, for example, PCT Pub. Nos. WO 2019/243293 and WO 2006/019095.

Guidelines for obtaining, making, purifying, and using an enzyme (e.g. an esterase or a carbamatase) are known in the art. To obtain enzymes for use as described in the present disclosure, the enzymes can be acquired commercially, isolated from an organism expressing the enzyme, or the enzyme can be produced through recombinant expression in a host cell and, optionally, isolated and purified for use in the methods described herein (e.g. cleavage of the linkers described herein).

Enzymatic reactions are carried out in an aqueous solution comprising a set of components. The set of components comprises the enzyme, a substrate, pH buffering additives, additives for obtaining a target ionic strength, cofactors (e.g. metal ions), detergents, and/or other additives suitable for enzyme activity and stability at a given temperature. Characterization of the enzymatic reaction (i.e. enzymatic activity) can be accomplished by measuring the products resulting from conversion of the substrate due to enzymatic activity and/or consumption of a cofactor or other additive due to enzymatic activity.

The nucleotide analogs described herein comprising a linker comprising an ester or a carbamate can be the substrate for the enzyme. Optimal conditions for linker cleavage can be identified by, for example, varying the concentrations of each of the components in the enzymatic reaction, the pH, and/or the temperature. Optimal conditions are defined by the method in which the linker cleavage is carried out. For example, it may be desirable to identify a set of conditions for the fastest possible reaction (e.g. linker cleavage) kinetics. A specific set of optimal conditions may be required for a given method in which the linker is to be cleaved. For example, cleaving a linker attaching a detectable label to the nucleotide analog in a method of sequencing can require a different set of optimal conditions than cleaving a linker attaching 3′-O-blocking moiety to the nucleotide analog in a method of synthesizing a nucleic acid. It is to be understood that optimal conditions are determined for each such method.

Guidelines for producing a carbamatase and determining conditions for activity can be found in, for example, Masaki et al. J Biosci Bioeng. 130(2):115-120. 2020; Liu et al. Mol Biotechnol. 59(2-3):84-97. 2017; and Zhou et al. Appl Biochem Biotechnol. 172(1):351-360. 2014, the contents of which are herein incorporated by reference in their entirety.

Guidelines for producing an esterase and determining conditions for activity can be found in, for example, Metin et al. J Basic Microbiol. 46(50):400-409. 2006; Deng et al. Apple Biochem Biotechnol. 176(1):1-12. 2015; Zheng et al. Protein Expr purif. 136:66-72. 2017; and Brod et al. Mol Biotechnol. 44(3):242-249. 2010, the contents of which are herein incorporated by reference in their entirety.

### Detectable Label

A label or detectable label, e.g., as bound to the reversible terminators described herein, may be any moiety that comprises one or more appropriate chemical substances or enzymes that directly or indirectly generate a detectable signal in a chemical, physical or enzymatic reaction. A large variety of labels are well known in the art. (See, e.g., International PCT Publication WO 2007/135368, “Dye Compounds and the Use of Their Labelled Conjugates,” incorporated by reference herein in its entirety).

For instance, one class of such labels is fluorescent labels. Fluorescent labels have the advantage of coming in several different wavelengths (colors) allowing distinguishably labeling each different terminator molecule. One example of such labels is dansyl-functionalized fluorescent moieties. Another example is the fluorescent cyanine-based labels Cy3 and Cy5, which can also be used in the present disclosure.

Other commercially available fluorescent labels include, but are not limited to, fluorescein and related derivatives such as isothiocyanate derivatives, e.g. FITC and TRITC, rhodamine, including TMR, texas red and Rox, bodipy, acridine, coumarin, pyrene, benzanthracene, the cyanins, succinimidyl esters such as NHS-fluorescein, maleimide activated fluorophores such as fluorescein-5-maleimide, phosphoramidite reagents containing protected fluorescein, boron-dipyrromethene (BODIPY) dyes, and other fluorophores, e.g. 6-FAM phosphoramidite 2. All of these types of fluorescent labels may be used in combination, in mixtures and in groups, as desired and depending on the application.

Various commercially available fluorescent labels are known in the art, such as Alexa Fluor Dyes, e.g., Alexa 488, 555, 568, 660, 532, 647, and 700 (Invitrogen-Life Technologies, Inc., California, USA, available in a wide variety of wavelengths, see for instance, Panchuk et al., J. Hist. Cyto., 47:1 179-1 188, 1999). Also commercially available are a large group of fluorescent labels called ATTO dyes (available from ATTO-TEC GmbH in Siegen, Germany). These fluorescent labels may be used in combinations or mixtures to provide distinguishable emission patterns for all terminator molecules used in the assay since so many different absorbance and emission spectra are commercially available.

Various commercially available fluorescent labels are known in the art, such as Alexa Fluor Dyes, e.g., Alexa 488, 555, 568, 660, 532, 647, and 700 (Invitrogen-Life Technologies, Inc., California, USA, available in a wide variety of wavelengths, see for instance, Panchuk, et al., J. Hist. Cyto., 47:1 179-1 188, 1999). Also commercially available are a large group of fluorescent labels called ATTO dyes (available from ATTO-TEC GmbH in Siegen, Germany). These fluorescent labels may be used in combinations or mixtures to provide distinguishable emission patterns for all terminator molecules used in the assay since so many different absorbance and emission spectra are commercially available.

In various exemplary embodiments, a label comprises a fluorescent dye, such as, but not limited to, a rhodamine dye, e.g., R6G, R1 10, TAN/IRA, and ROX, a fluorescein dye, e.g., JOE, VIC, TET, HEX, FAM, etc., a halo-fluorescein dye, a cyanine dye. e.g., CY3, CY3.5, CY5, CY5.5, etc., a BODIPY® dye, e.g., FL, 530/550, TR, TMR, etc., a dichlororhodamine dye, an energy transfer dye, e.g., BIGDYE™ v 1 dyes, BIGDYE™ v 2 dyes, BIGDYE™ v 3 dyes, etc., Lucifer dyes, e.g., Lucifer yellow, etc., CASCADE BLUE®, Oregon Green, and the like. Other exemplary dyes are provided in Haugland, Molecular Probes Handbook of Fluorescent Probes and Research Products, Ninth Ed. (2003) and the updates thereto. Non-limiting exemplary labels also include, e.g., biotin, weakly fluorescent labels (see, for instance, Yin et al., Appl Environ Microbiol., 69(7):3938, 2003; Babendure et al., Anal. Biochem., 317(1): 1, 2003; and Jankowiak et al., Chem. Res. Toxicol., 16(3):304, 2003), non-fluorescent labels, colorimetric labels, chemiluminescent labels (see, Wilson et al., Analyst, 128(5):480, 2003; Roda et al., Luminescence, 18(2):72, 2003), Raman labels, electrochemical labels, bioluminescent labels (Kitayama et al., Photochem. Photobiol., 77(3):333, 2003; Arakawa et al., Anal. Biochem., 314(2): 206, 2003; and Maeda, J. Pharm. Biomed. Anal., 30(6): 1725, 2003), and the like.

Multiple labels can also be used in the disclosure. For example, bi-fluorophore FRET cassettes (Tet. Letts., 46:8867-8871, 2000) are well known in the art and can be utilized in the disclosed methods. Multi-fluor dendrimeric systems (J. Amer. Chem. Soc., 123:8101-8108, 2001) can also be used. Other forms of detectable labels are also available. For example, microparticles, including quantum dots (Empodocles et al., Nature, 399:126-130, 1999), gold nanoparticles (Reichert et al., Anal. Chem., 72:6025-6029, 2000), microbeads (Lacoste et al., Proc. Natl. Acad. Sci. USA, 97(17):9461-9466, 2000), and tags detectable by mass spectrometry can all be used.

Multi-component labels can also be used in the disclosure. A multi-component label is one which is dependent on the interaction with a further compound for detection. The most common multi-component label used in biology is the biotin-streptavidin system. Biotin is used as the label attached to the nucleotide base. Streptavidin is then added separately to enable detection to occur. Other multi-component systems are available. For example, dinitrophenol has a commercially available fluorescent antibody that can be used for detection.

Thus, a “label” as presently defined is a moiety that facilitates detection of a molecule. Common labels in the context of the present disclosure include fluorescent, luminescent, light-scattering, and/or colorimetric labels. Suitable labels may also include radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241, each incorporated by reference in its entirety. As other non-limiting examples, the label can be a luminescent label, a light-scattering label (e.g., colloidal gold particles), or an enzyme (e.g., Horse Radish Peroxidase (HRP)).

Fluorescence energy transfer (FRET) dyes may also be employed, such as DY-630/DY-675 from Dyomics GmbH of Germany, which also commercially supplies many different types of dyes including enzyme-based labels, fluorescent labels, etc. (See, for instance, Dohm et al., “Substantial biases in ultra-short read data sets from high-throughput DNA sequencing,” Nucleic Acids Res., 36:e105, 2008).

The label and linker construct can be of a size or structure sufficient to act as a block to the incorporation of a further nucleotide onto the nucleotide of the disclosure. This permits controlled polymerization to be carried out. The block can be due to steric hindrance, or can be due to a combination of size, charge and structure.

### Modified Nucleotide Synthesis

In some embodiments, the linker is attached the 5 position of pyrimidines or the 7 position of 7-deazapurines. In other embodiments, the linker may be attached to an exocyclic amine of a nucleobase, e.g. by N-alkylating or N-acylating the exocyclic amine of cytosine. In other embodiments, the linker may be attached to any other atom in the nucleobase.

Certain polymerases have a high tolerance for modification of certain parts of a nucleotide, e.g. modifications of the 5 position of pyrimidines and the 7 position of purines are well-tolerated by some polymerases (He and Seela., Nucleic Acids Research 30.24 (2002): 5485-5496.; or Hottin et al., Chemistry. 2017 Feb. 10; 23(9):2109-2118). In some embodiments, the linker is attached to these positions.

In some examples, a labeled nucleotide is prepared by first synthesizing an intermediate compound comprising a linker and a nucleotide (referred to herein as a “linker-nucleotide”), and then this intermediate compound is attached to the label. In some examples, nucleotides with substitutions compared to natural nucleotides, e.g. pyrimidines with 5-hydroxymethyl or 5-propargylamino substituents, or 7-deazapurines with 7-hydroxymethyl or 7-propargylamino substituents may be useful starting materials for preparing linker-nucleotides. An exemplary set of nucleotides with 5- and 7-hydroxymethyl substituents that may be useful for preparing linker-nucleotides is shown.

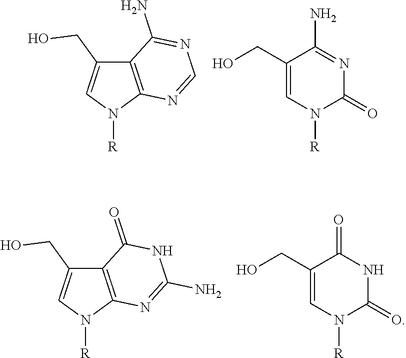

An exemplary set of nucleotides with 5- and 7-deaza-7-propargylamino substituents that may be useful for preparing linker-nucleotides is shown below:

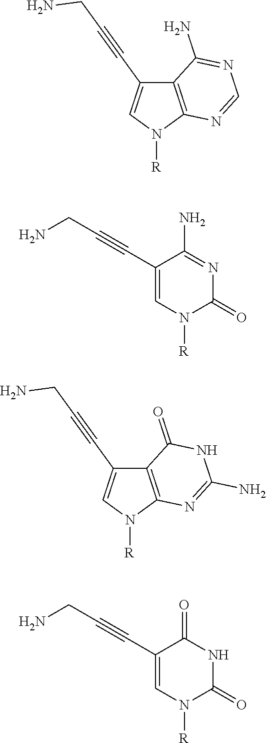

These nucleotides are also commercially available as deoxyribonucleoside triphosphates.

### Methods of Stepwise Polynucleotide Synthesis

In another aspect, the present disclosure provides a method of sequencing a single-stranded polynucleotide, comprising
a) incorporating the nucleotide analog provided herein into a primer hybridized to said single-stranded polynucleotide using a polymerase;
b) detecting the identity of said nucleotide analog; and
c) contacting said nucleic acid molecule with an esterase or an carbamatase.

In some embodiments, said esterase or carbamatase reacts with said incorporated nucleotide analog to expose a 3′ OH group.

In other embodiments, said esterase or carbamatase reacts with said incorporated nucleotide analog to cleave a linker bound to said base.

In certain embodiments, said incorporating is accomplished via a polymerase.

In some embodiments, said nucleotide analog comprises or is bound to a label.

In other embodiments, detecting the identity of said nucleotide analog comprises detecting said label.

The nucleotide analogs provided herein can be used in any method of nucleic acid synthesis known in the art comprising the use of a 3′-O-blocked, or reversibly blocked 3′-O-blocked nucleotide analog (i.e. reversible terminator). Illustrative examples of such nucleic acid synthesis methods can be found in, for example, PCT Pub. Nos. WO 2018/119253, WO 2020/141143, WO 2021/122539, WO 2015/175832, WO 2021/045830, WO 2017/142913, WO 2017/184677, WO 2018/102554, WO 2018/102818, WO 1996/007669, WO 2021/094251, WO 2018/215803, WO 2016/139477, WO 2018/138508, WO 2019/053443, WO 2020/178603, WO 2020/229831, WO 2020/234605, WO 2021/148809, WO 2019/224544, WO 2020/016606, WO 2020/016604, WO 2020/016605, WO 2021/048545, U.S. Pat. No. 11,117,922, and US Pub. No. 2021/0130863, the contents of which are herein incorporated by reference in their entirety.

In another aspect, the present disclosure provides a method of labeling a nucleic acid molecule, comprising
a) incorporating the nucleotide analog provided herein into the nucleic acid molecule, wherein said nucleotide analog comprises or is bound to a label; and
b) contacting the nucleic acid molecule with an esterase or an carbamatase.

In some embodiments, the method further comprises detecting the identity of said label.

In other embodiments, said esterase or carbamatase reacts with said incorporated nucleotide analog to expose a 3′ OH group.

In certain embodiments, said esterase or carbamatase reacts with said incorporated nucleotide analog to cleave a linker bound to said base.

In some embodiments, said incorporating is accomplished via a polymerase.

In other embodiments, the method further comprises detecting the identity of the label before contacting said nucleic acid molecule with said esterase or carbamatase.

In another aspect, the present disclosure provides of synthesizing a single-stranded polynucleotide, comprising binding the nucleotide analog provided herein to the 3′ hydroxyl end of a polynucleotide.

In some embodiments, said binding of said nucleotide analog to said polynucleotide is catalyzed using a polymerase. In some embodiments, the polymerase is a template-independent polymerase.

In other embodiments, said esterase or carbamatase reacts with said incorporated nucleotide analog to expose a 3′ OH group of said nucleotide analog.

In certain embodiments, the method further comprises contacting said nucleotide analog bound to said single-stranded polynucleotide with an esterase or an carbamatase, wherein said esterase or carbamatase reacts with said nucleotide analog to expose the 3′ OH group of said nucleotide analog.

In some embodiments, the method further comprises repeating said binding and contacting with said esterase or said carbamatase steps.

In other embodiments, the single-stranded polynucleotide is immobilized on a solid support. In some embodiments, the nucleotide analog comprises or is bound to a label, further comprising detecting the identity of said label.

### Polymerase

In certain embodiments, the polymerase is a DNA polymerase. In some embodiments, the polymerase is an RNA polymerase. In other embodiments, the polymerase is a template-independent polymerase. In some embodiments, the polymerase is a template-dependent polymerase.

In some embodiments, the template-independent polymerase is Terminal Deoxynucleotidyl Transferase (TdT) or a variant thereof. In some embodiments, the template-independent polymerase must have a DNA nuclotidylexotransferase activity. In other embodiments, the template-independent polymerase is Polymerase Theta, which has template-independent activity under certain conditions. In some embodiments, the catalytic activity of the template-independent polymerase is found under Enzyme Commision number EC 2.7.7.31. In other embodiments, the template-independent polymerase is an RNA polymerase such as polynucleotide adenylyltransferase (EC 2.7.7.19) or polynucleotide uridylyltransferase (EC 2.7.7.52) or variant thereof. Illustrative wild type TdT and TdT variants can be found in, for example, PCT App. Nos. WO 2001/064909, WO 2018/217689, WO 2018/215803, WO 2020/072715, WO 2020/081985, WO 2020/099451, WO 2020/161480, WO 2020/239737, WO 2021/094251, WO 2021/116270, and U.S. Pat. No. 7,494,797, the contents of which are herein incorporated by reference in their entirety.

In some embodiments, the template-dependent polymerase is a DNA-directed DNA polymerase (which terms are used interchangeably to refer to an enzyme having activity 2.7.7.7 using the IUBMB nomenclature), or an DNA-directed RNA polymerase. A description of such enzymes can be found in Richardson, A. Enzymatic synthesis of deoxyribonucleic acid. XIV. Further purification and properties of deoxyribonucleic acid polymerase of Escherichia coli. J. Biol. Chem. 239 (1964) 222-232; Schachman, A. Enzymatic synthesis of deoxyribonucleic acid. VII. Synthesis of a polymer of deoxyadenylate and deoxythymidylate. J. Biol. Chem. 235 (1960) 3242-3249; and Zimmerman, B. K. Purification and properties of deoxyribonucleic acid polymerase from Micrococcus lysodeikticus. J. Biol. Chem. 241 (1966) 2035-2041.

To achieve the presently claimed methods, polymerase enzymes must be selected which are tolerant of modifications of the nucleotide analog molecule disclosed herein. Such tolerant polymerases tolerant to modifications at the 3′ end and to the base are known and commercially available.

Mutant forms of 9° N-7(exo-) DNA polymerase can further improve tolerance for such modifications (WO 2005024010; WO 2006120433), while maintaining high activity and specificity. An example of a suitable polymerase is THERMINATOR™ DNA polymerase (New England Biolabs, Inc., Ipswich, MA), a Family B DNA polymerase, derived from Thermococcus species 9° N-7. The 9° N-7(exo-) DNA polymerase contains the D141A and E143A variants causing 3′-5′ exonuclease deficiency. (See, Southworth et al., “Cloning of thermostable DNA polymerase from hyperthermophilic marine Archaea with emphasis on Thermococcus species 9° N-7 and mutations affecting 3′-5′ exonuclease activity,” Proc. Natl. Acad. Sci. USA, 93(1 1): 5281-5285, 1996). THERMINATOR™ I DNA polymerase is 9° N-7(exo-) that also contains the A485L variant. (See, Gardner et al, “Acyclic and dideoxy terminator preferences denote divergent sugar recognition by archaeon and Taq DNA polymerases,” Nucl. Acids Res., 30:605-613, 2002). THERMINATOR™ III DNA polymerase is a 9° N-7(exo-) enzyme that also holds the L408S, Y409A and P410V mutations. These latter variants exhibit improved tolerance for nucleotides that are modified on the base and 3′ position. Another polymerase enzyme useful in the present methods and with the reversible terminators described herein is the exo-mutant of KOD DNA polymerase, a recombinant form of Thermococcus kodakaraensis KOD1 DNA polymerase. (See, Nishioka et al., “Long and accurate PCR with a mixture of KOD DNA polymerase and its exonuclease deficient mutant enzyme,” J. Biotech., 88:141-149, 2001). The thermostable KOD polymerase is capable of amplifying target DNA up to 6 kbp with high accuracy and yield. (See, Takagi et al., “Characterization of DNA polymerase from Pyrococcus sp. strain KOD1 and its application to PCR,” App. Env. Microbiol., 63(1 1):4504-4510, 1997). Others are Vent (exo-), Tth Polymerase (exo-), and Pyrophage (exo-) (available from Lucigen Corp., Middletown, WI, US). Another non limiting exemplary DNA polymerase is the enhanced DNA polymerase, or EDP (See, WO 2005/024010).

When sequencing using SBE, suitable DNA polymerases include, but are not limited to, the Klenow fragment of DNA polymerase I, SEQUENASE™ 1.0 and SEQUENASE™ 2.0 (U.S. Biochemical), T5 DNA polymerase, Phi29 DNA polymerase, THERMO SEQUENASE™ (Taq polymerase with the Tabor-Richardson mutation, see Tabor et al., Proc. Natl. Acad. Sci. USA, 92:6339-6343, 1995) and others known in the art or described herein. Modified versions of these polymerases that have improved ability to incorporate a nucleotide analog of the disclosure can also be used. Further, it has been reported that altering the reaction conditions of polymerase enzymes can impact their promiscuity, allowing incorporation of modified bases and reversible terminator molecules. For instance, it has been reported that addition of specific metal ions, e.g., Mn²⁺, to polymerase reaction buffers yield improved tolerance for modified nucleotides, although at some cost to specificity (error rate). Additional alterations in reactions may include conducting the reactions at higher or lower temperature, higher or lower pH, higher or lower ionic strength, inclusion of co-solvents or polymers in the reaction, and the like.

Random or directed mutagenesis may also be used to generate libraries of mutant polymerases derived from native species; and the libraries can be screened to select mutants with optimal characteristics, such as improved efficiency, specificity and stability, pH and temperature optimums, and the like.

### Equivalents and Scope

Those skilled in the art will recognize, or be able to ascertain using no more than routine experimentation, many equivalents to the specific embodiments in accordance with the nucleotide analogs and methods described herein. The scope of the present disclosure is not intended to be limited to the Description provided herein, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments provided in the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control. Section and table headings are not intended to be limiting.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

The following terms are intended to have the meanings presented therewith below and are useful in understanding the description and intended scope of the present disclosure. It should be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue (i.e. analog)” means one analogue or more than one analogue.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The disclosure is not intended to be limited in any manner by the above exemplary listing of substituents.

### EXAMPLES

Below are examples of specific embodiments for making, using and characterizing the nucleotide analogs and methods disclosed herein. The examples are offered for illustrative purposes only, and are not intended to limit the scope of the disclosure in any way. Efforts have been made to ensure accuracy with respect to numbers used (e.g., amounts, temperatures, etc.), but some experimental error and deviation should, of course, be allowed for.

The practice of the nucleotide analogs and methods disclosed herein will employ, unless otherwise indicated, conventional methods of organic chemistry, protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1: Synthesis of Nucleotide Analogs

### Stable 3′ Esters

The synthetic route illustrated in Scheme 1 depicts an exemplary procedure for preparing 3′ ester dTTP analog. In the first step, thymidine is selectively protected at 5′-OH with TBS group, then it undergoes ester formation at 3′-OH with the carboxylic acid R₁—COOH with DCC and DMAP. After the 5′-O-TBS group is removed in the third step, the nucleoside is finally converted into the triphosphate using the “one-pot, three-step” method, starting with monophosphorylation of nucleoside followed by reaction with tributylammonium pyrophosphate and hydrolysis of the resulting cyclic intermediate to provide the corresponding dNTP. In a preferred embodiment, R₁=1-methyl-1-cyclopropyl.

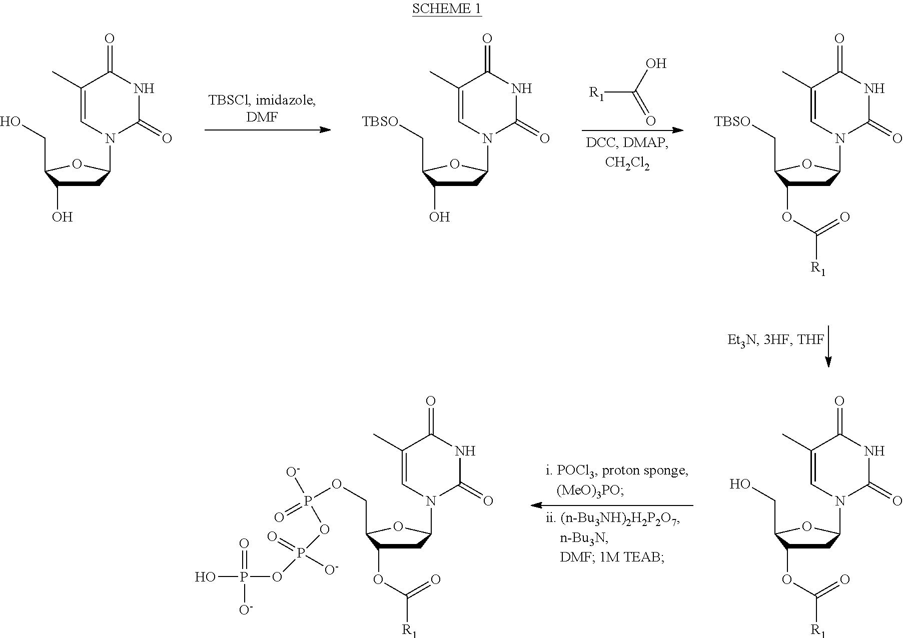

The synthetic route illustrated in Scheme 2 depicts an exemplary procedure for preparing 3′ ester dCTP analog. In the first step, 2′-deoxycytidine is selectively protected at 5′-OH with DMTr group, then it undergoes ester formation at 3′-OH with the carboxylic acid R₁—COOH with DCC and DMAP. After the 5′-O-DMTr group is removed in the third step, the nucleoside is finally converted into the triphosphate using the “one-pot, three-step” method, starting with monophosphorylation of nucleoside followed by reaction with tributylammonium pyrophosphate and hydrolysis of the resulting cyclic intermediate to provide the corresponding dNTP. In a preferred embodiment, R₁=1-methyl-1-cyclopropyl.

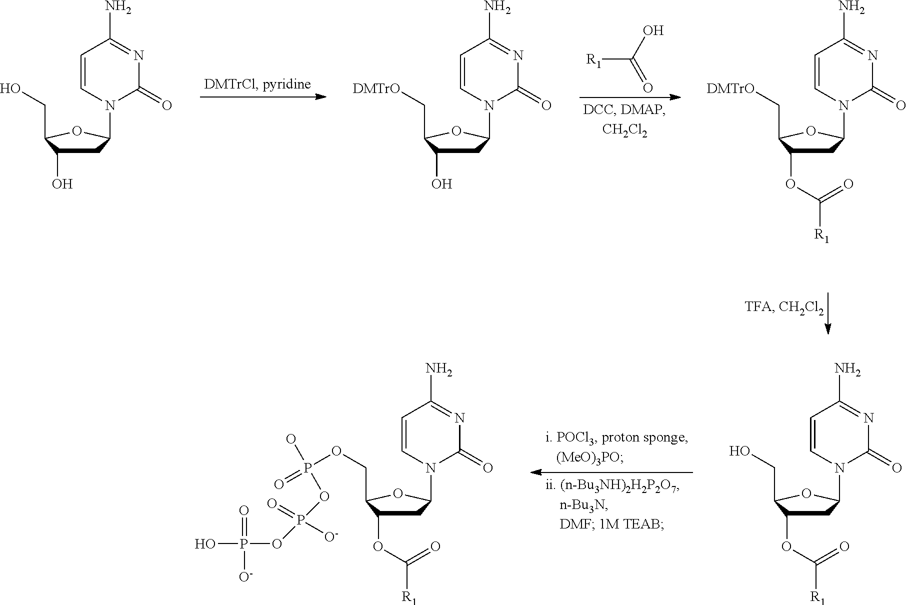

The synthetic route illustrated in Scheme 3 depicts an exemplary procedure for preparing 3′ ester dATP analog. In the first step, 2′-deoxyadenosine is selectively protected at 5′-OH with TBS group, then it undergoes ester formation at 3′-OH with the carboxylic acid R₁—COOH with DCC and DMAP. After the 5′-O-TBS group is removed in the third step, the nucleoside is finally converted into the triphosphate using the “one-pot, three-step” method, starting with monophosphorylation of nucleoside followed by reaction with tributylammonium pyrophosphate and hydrolysis of the resulting cyclic intermediate to provide the corresponding dNTP. In a preferred embodiment, R₁=1-methyl-1-cyclopropyl.

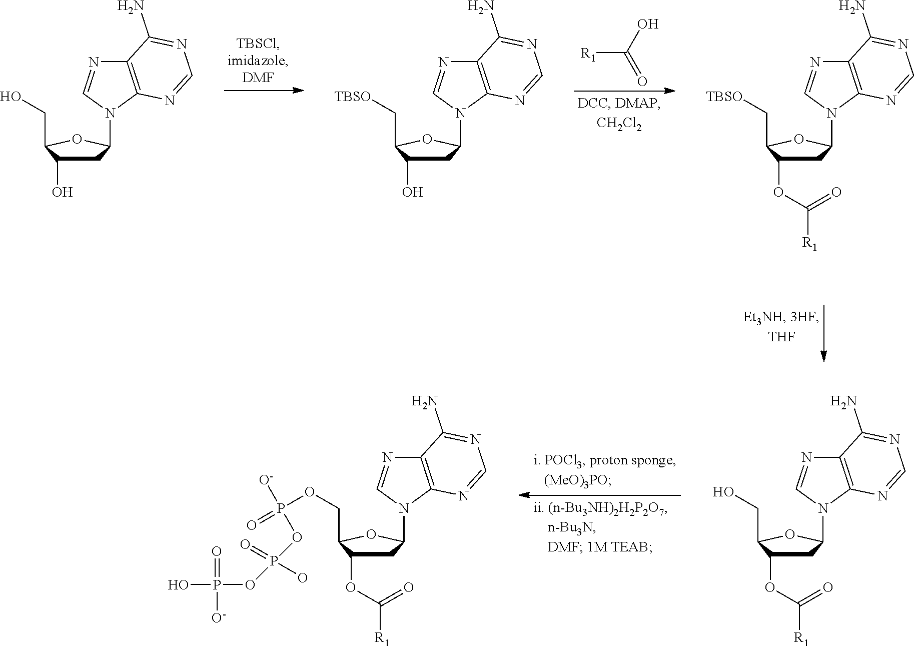

The synthetic route illustrated in Scheme 4 depicts an exemplary procedure for preparing 3′ ester dGTP analog. In the first step, 2′-deoxyguanosine is selectively protected at 5′-OH with TBS group, then it undergoes ester formation at 3′-OH with the carboxylic acid R₁—COOH with DCC and DMAP. After the 5′-O-TBS group is removed in the third step, the nucleoside is finally converted into the triphosphate using the “one-pot, three-step” method, starting with monophosphorylation of nucleoside followed by reaction with tributylammonium pyrophosphate and hydrolysis of the resulting cyclic intermediate to provide the corresponding dNTP. In a preferred embodiment, R₁=1-methyl-1-cyclopropyl.

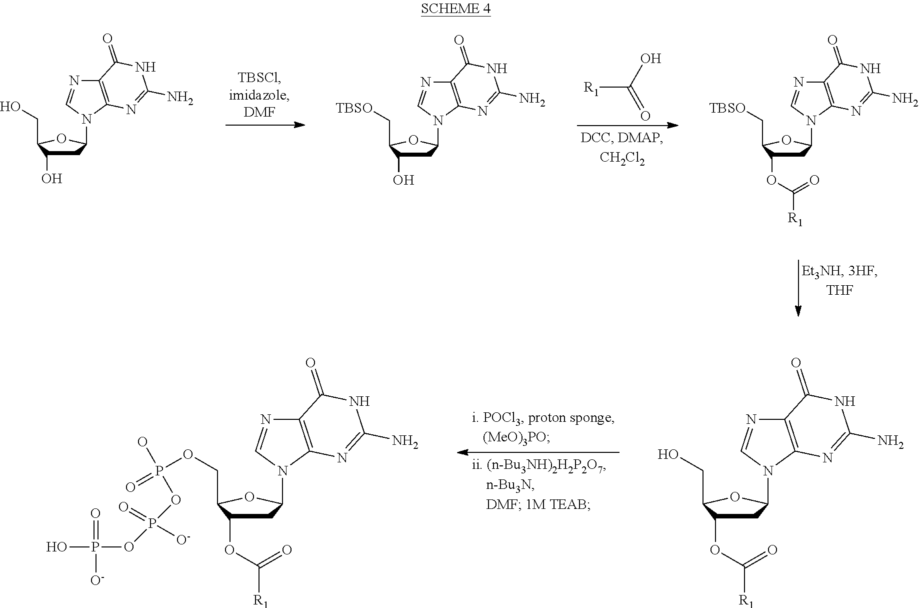

### 3′ Carbamates

The synthetic route illustrated in Scheme 5 depicts an exemplary procedure for preparing 3′ carbamoyl dCTP analog. In the first step, N4-benzoyl-5′-O-DMT-2′-deoxythymidine undergoes carbonate formation at the 3′-OH with 4-nitrophenyl chloroformate. Next, the 4-nitrophenyl carbonate is treated with the amine R₂NHR₃ to convert into the 3′-O-carbamoyl nucleoside. Finally, the 5′-O-DMT group is removed, and the nucleoside is phosphorylated into triphosphate using the “one-pot, three-step” method, starting with monophosphorylation of nucleoside followed by reaction with tributylammonium pyrophosphate and hydrolysis of the resulting cyclic intermediate to provide the corresponding dNTP, and the benzoyl group is removed using ammonium hydroxide. In a preferred embodiment, R₂═R₃═H.

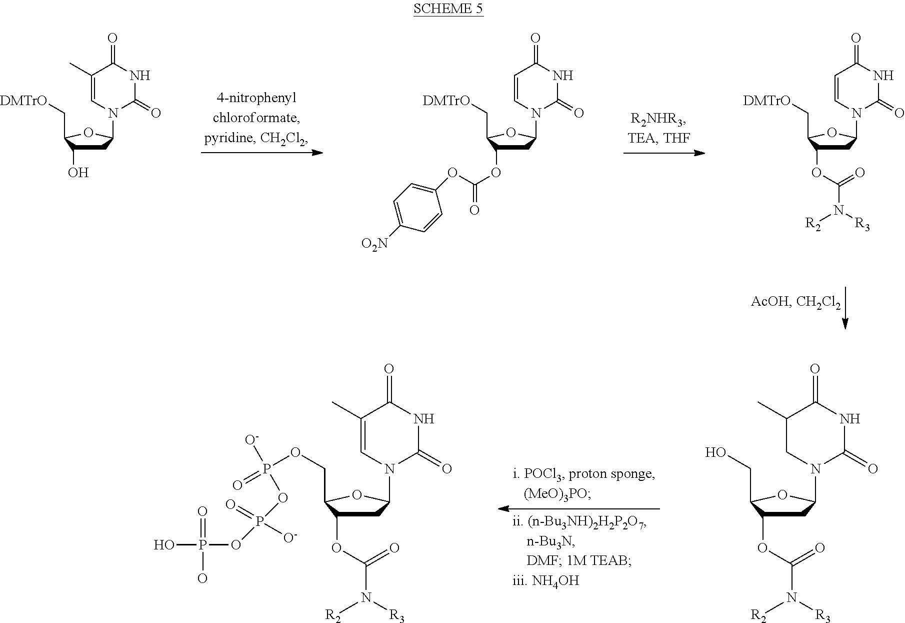

The synthetic route illustrated in Scheme 6 depicts an exemplary procedure for preparing 3′ carbamoyl dCTP analog. In the first step, N4-benzoyl-5′-O-DMT-2′-deoxycytidine undergoes carbonate formation at the 3′-OH with 4-nitrophenyl chloroformate. Next, the 4-nitrophenyl carbonate is treated with the amine R₂NHR₃ to convert into the 3′-O-carbamoyl nucleoside. Finally, the 5′-O-DMT group is removed, and the nucleoside is phosphorylated into triphosphate using the “one-pot, three-step” method, starting with monophosphorylation of nucleoside followed by reaction with tributylammonium pyrophosphate and hydrolysis of the resulting cyclic intermediate to provide the corresponding dNTP, and the benzoyl group is removed using ammonium hydroxide. In a preferred embodiment, R₂═R₃═H.

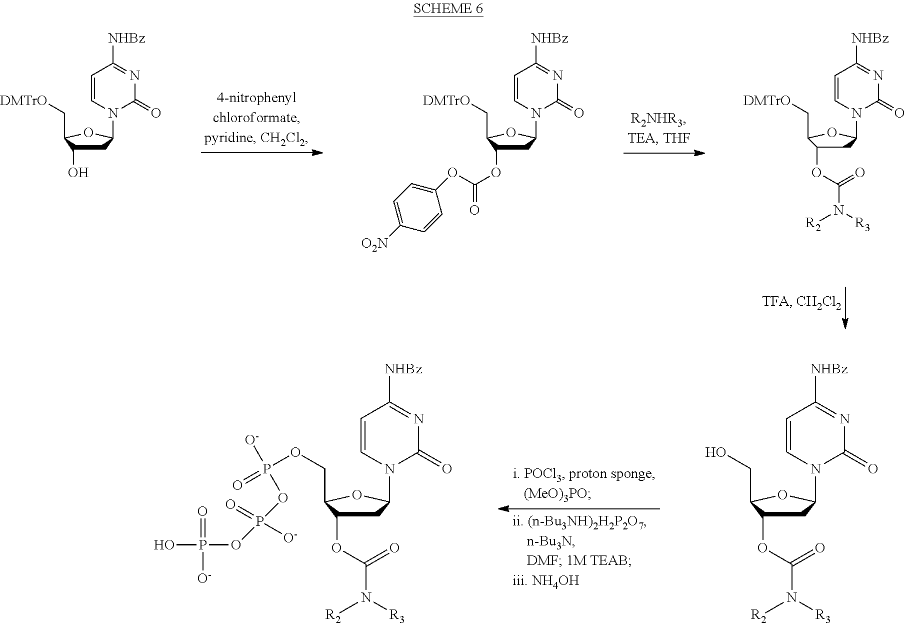

The synthetic route illustrated in Scheme 7 depicts an exemplary procedure for preparing 3′ carbamoyl dATP analog. In the first step, N6-benzoyl-5′-O-DMT-2′-deoxyadenosine undergoes carbonate formation at the 3′-OH with 4-nitrophenyl chloroformate. Next, the 4-nitrophenyl carbonate is treated with the amine R₂NHR₃ to convert into the 3′-O-carbamoyl nucleoside. Finally, the 5′-O-DMT group is removed, and the nucleoside is phosphorylated into triphosphate using the “one-pot, three-step” method, starting with monophosphorylation of nucleoside followed by reaction with tributylammonium pyrophosphate and hydrolysis of the resulting cyclic intermediate to provide the corresponding dNTP, and the benzoyl group is removed using ammonium hydroxide. In a preferred embodiment, R₂═R₃═H.

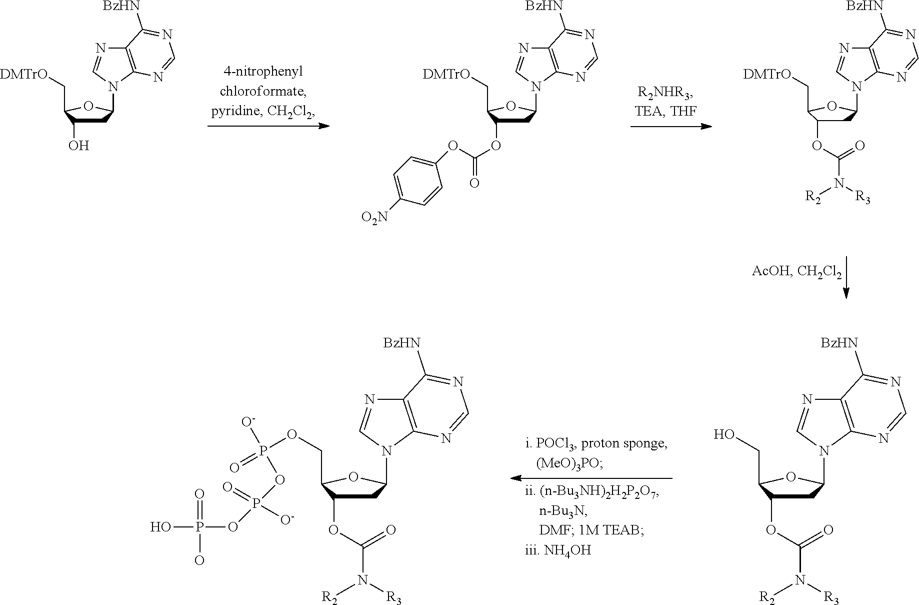

The synthetic route illustrated in Scheme 8 depicts an exemplary procedure for preparing 3′ carbamoyl dGTP analog. In the first step, N2-isobutyryl-5′-O-DMT-2′-deoxyguanosine undergoes carbonate formation at the 3′-OH with 4-nitrophenyl chloroformate. Next, the 4-nitrophenyl carbonate is treated with the amine R₂NHR₃ to convert into the 3′-O-carbamoyl nucleoside. Finally, the 5′-O-DMT group is removed, and the nucleoside is phosphorylated into triphosphate using the “one-pot, three-step” method, starting with monophosphorylation of nucleoside followed by reaction with tributylammonium pyrophosphate and hydrolysis of the resulting cyclic intermediate to provide the corresponding dNTP, and the isobutyryl group is removed using ammonium hydroxide. In a preferred embodiment, R₂═R₃═H.

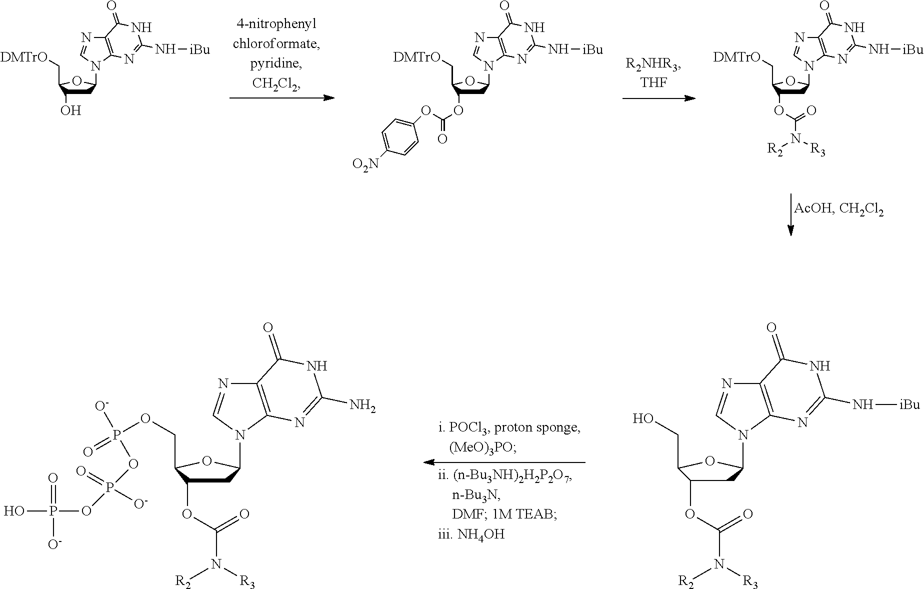

A nucleotide analog of Formula (I-A) may be prepared using procedures similar to the general synthetic protocols described above.

### Example 2: Enzymatic Incorporation/Polynucleotide Synthesis

Cyclic synthesis of a DNA sequence. Four cycles consisting of an Extension and Deprotection step are performed on an initiator oligonucleotide. The initator oligonucleotide has the sequence T₃₅ and is modified on the 5′ end with a 6-carboxyfluorescein label to facilitate size analysis by capillary electrophoresis. In each extension step, the purified oligonucleotide is exposed to a Terminal Deoxynucleotidyl Transferase enzyme and a 3′ reversible terminator dNTP described in Example 1 in Extension Reaction Buffer containing 20 mM Tris Acetate, 50 mM Potassium Acetate, 10 mM Magnesium Acetate, and 0.25 mM Cobalt Chloride, pH 7.9, at 37° C. for 1 hour. The reaction is quenched by addition of EDTA to a final concentration of 100 mM, and the oligonucleotide is purified using the Oligo Clean and Concentrator Kit (Zymo Research) and is eluted from the column using deionized water. A portion of the purified oligonucleotide extension product is set aside for capillary electrophoresis. The remainder of the purified extension product is used in the subsequent Deprotection step. Purified extension product is added to the Deprotection reaction containing an esterase or carbamatase that can cleave the terminator group in a compatible buffer for a sufficient duration to completely remove the terminator. The reaction products are purified using the Oligo Clean and Concentrator Kit and are eluted from the column using deionized water. A portion of the purified oligonucleotide extension product is set aside for size analysis by capillary electrophoresis. Four cycles Extension and Deprotection are performed, and the purified oligonucleotide products are analyzed by capillary electrophoresis, diluting with 3 volumes of with HiDi formamide (ThermoFisher Scientific) containing GeneScan Liz600 (ThermoFisher) size standards and run on an ABI 3730xl DNA Analyzer in “Fragment Analysis” mode. Capillary electropherograms of the initiator and reaction products are done to show that the initiator is elongated by one nucleotide per cycle, totaling four nucleotides for the final product.

Instead of performing these reactions in solution with DNA purification in between each step, the reactions described above are also performed with the oligonucleotide immobilized on a solid support, with purification replaced by washing. Exemplary solid supports include a magnetic bead, a resin, and the inner surface of a flow cell.

To demonstrate incorporation of an illustrative nucleotide analog described herein, 2 Units/uL Murine TdT (obtained from New England BioLabs) was incubated in 20 mM Tris Acetate pH 7.9, 10 mM Magnesium Acetate, 50 mM Potassium Acetate, 100 μg/mL Bovine, 50 nM of a 5′-6-FAM labeled initiator oligo (with the sequence:

5′-CTGACAGAGATGATGAAGTCACATGAGACATGAACTGAGTCTTTT-3′

(SEQ ID NO: 1)) and 1 mM nucleotide analog. Two nucleotide analogs of dTTP having a carbamate linker attached to the 3′ position of the ribose suger were tested. The carbamate containing linker had either a terminal carbamate group or a terminal methyl group. The extension (i.e. incorporation) reaction was incubated for 16 hours at 37° C. Subsequently, 1 mM dATP was added to this reaction to extend any products with unblocked 3′-OH ends, and therefore allow differentiation of extension products containing the nucleotide analog having the carbamate containing linker from those having unblocked 3′-OH ends when characterized by capillary electrophoresis.

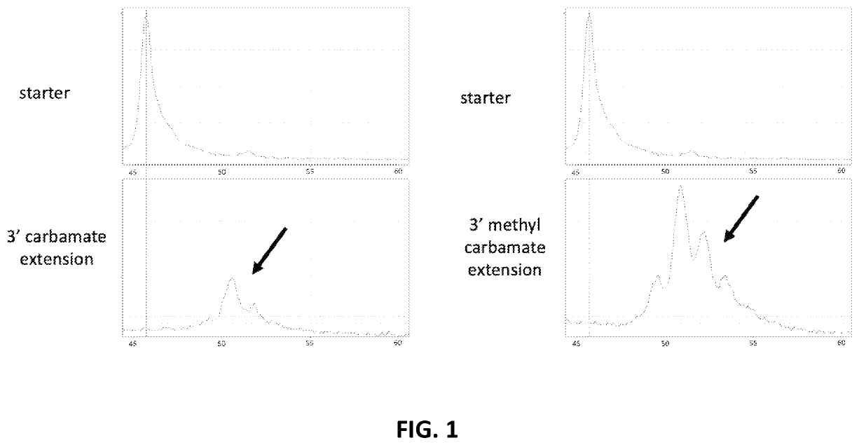

Oligonucleotide products were analyzed by capillary electrophoresis (FIG. 1). The initiator oligo peak is demarcated with an vertical dashed line. Arrows denote capillary electrophoresis peaks with initiator oligo having a dTTP nucleotide analog comprising a carbamate containing linker incorporated following the extension reaction. The results in FIG. 1 show that the dTTP nucleotide analogs having a carbamate containing linker were incorporated into initiator oligo by TdT. Furthermore, the results indicate that the carbamate containing linker was able to prevent further extension by dATP in both dTTP analogs tested.

### Example 3: Sequence Detection

Sequencing of a target polynucleotide is carried out by contacting a target polynucleotide separately with different modified nucleotides described herein to form the complement to that of the target polynucleotide and detecting the incorporation of the modified nucleotide.

For each cycle, a nucleotide is incorporated into a target polynucleotide by a polymerase enzyme. Examples of polymerase enzymes suitable for incorporation include DNA polymerase I, the Klenow fragment, DNA polymerase III, T4 or T7 DNA polymerase, Taq polymerase or vent polymerase. A polymerase engineered to have specific properties to incorporate the modified nucleotides described herein can also be used.

To carry out the polymerase reaction, a primer sequence is annealed to the target polynucleotide, the primer sequence being recognised by the polymerase enzyme and acting as an initiation site for the subsequent extension of the complementary strand. Other conditions necessary for carrying out the polymerase reaction, including temperature, pH, buffer compositions etc., will be apparent to those skilled in the art.

The modified nucleotides of the disclosure are brought into contact with the target polynucleotide, to allow polymerisation to occur. The nucleotides may be added sequentially, i.e., separate addition of each nucleotide type (A, T, G or C), or added together. If they are added together, each nucleotide type will be labelled with a unique label.

This polymerisation step is allowed to proceed for a time sufficient to allow incorporation of a nucleotide.

Nucleotides that are not incorporated are then removed, for example, by a washing step. Detection of the incorporated labels may then be carried out.

After detection, the label is removed by adding carbamatase to cleave the linker and remove the reversible terminator.

The above steps will be repeated for each cycle to obtain further sequence information.

It is to be understood that the words which have been used are words of description rather than limitation, and that changes may be made within the purview of the appended claims without departing from the true scope and spirit of the disclosure in its broader aspects.

While the nucleotide analogs and methods of the disclosure have been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A nucleotide analog of the formula (1-A): 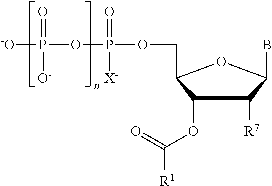
R¹ is selected from the group consisting of: 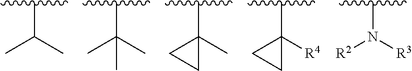
R², R³ and R⁴ are each is selected from the group consisting of: H, CH₃, and CH₂CH₃;
X is O or S;
R⁷ is selected from H, OH, F, OMe, and —O-2-methoxyethyl;
n is 0, 1, 2, 3, 4 or 5; and
B is a nucleotide base or an analog thereof.

2. A nucleotide analog of formula (I-B): 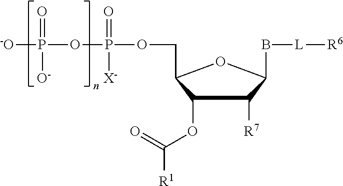
R¹ is selected from the group consisting of: 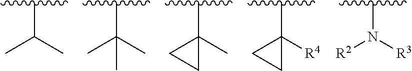
R², R³ and R⁴ are each selected from the group consisting of: H, CH₃ and CH₂CH₃;
X is O or S;
R⁷ is selected from H, OH, F, OMe, and —O-2-methoxyethyl;
n is 0, 1, 2, 3, 4 or 5;
B is a nucleotide base or an analog thereof;
L is a linker comprising a cleavable linkage; and
R⁶ is a label; wherein R⁶ and B are covalently linked via L.

3. The nucleotide analog of claim 1 or 2, wherein B is a nucleotide base.

4. The nucleotide analog of claim 1, wherein B is a scarred nucleotide base.

5. The nucleotide analog of claim 4, wherein the scarred nucleotide base is a nucleotide base substituted with —CH₂—OH, —C—C—CH₂—OH, or —C—C—CH₂—NHC(O)CH₂—OH.

6. The nucleotide analog of claim 4 or 5, wherein the scarred nucleotide base is selected from

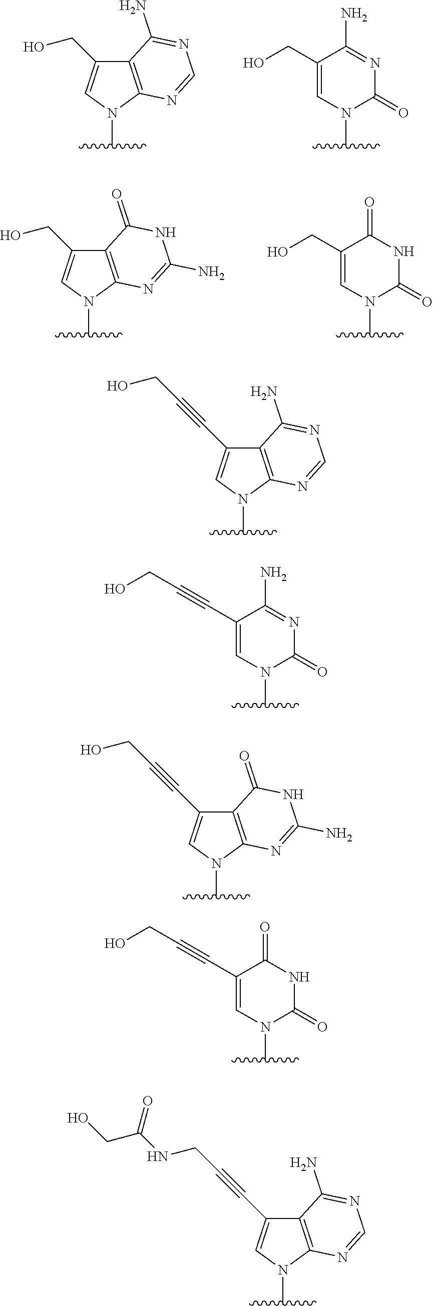

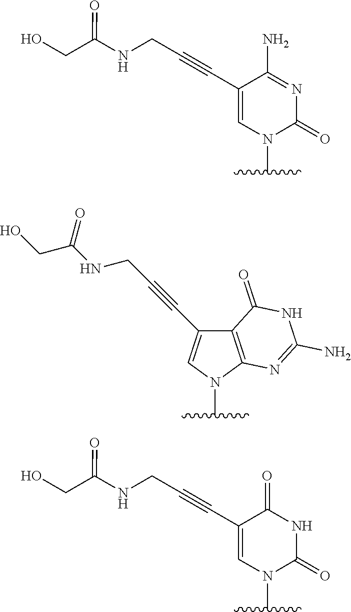 [SMILES: *n1cc(C#CCNC(=O)CO)c(N)nc1=O ; *n1cc(C#CCNC(=O)CO)c2c(=O)[nH]c(N)nc21 ; *n1cc(C#CCNC(=O)CO)c(=O)[nH]c1=O]

7. The nucleotide analog of any one of claims 1-6, wherein X is O.

8. The nucleotide analog of any one of claims 1-7, wherein R², R³ and R⁴ are each selected from the group consisting of: H and CH₃.

9. The nucleotide analog of any one of claims 1-8, wherein R² is H and R³ is H.

10. The nucleotide analog of any one of claims 1-8, wherein R² is H and R³ is CH₃.

11. The nucleotide analog of any one of claims 1-8, wherein R² is CH₃ and R₃ is H.

12. The nucleotide analog of any one of claims 1-8, wherein R² is CH₃ and R₃ is CH₃.

13. The nucleotide analog of any one of claims 9-12, wherein R⁴ is H.

14. The nucleotide analog of any one of claims 9-12, wherein R⁴ is CH₃.

15. The nucleotide analog of any one of claims 1-14, wherein R⁷ is H.

16. The nucleotide analog of any one of claims 1-15, wherein n is 0.

17. The nucleotide analog of any one of claims 1-15, wherein n is 1.

18. The nucleotide analog of any one of claims 1-15, wherein n is 2.

19. The nucleotide analog of any one of claims 2-18, wherein L is -L²-L¹-, wherein:
L¹ is a bond or a linkage group comprising a hydrocarbon and optionally other atoms (e.g., N, O and S) and L¹ is attached to R⁶; and
L² is selected from the group consisting of: 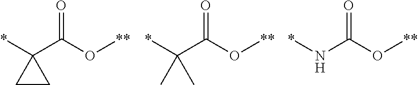
wherein * denotes the point of attachment to L¹ and ** denotes the point of attachment to B.

20. The nucleotide analog of claim 19, wherein L¹ is a polypeptide.

21. The nucleotide analog of claim 19 or 20, wherein L² is:

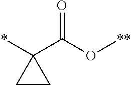

22. The nucleotide analog of claim 19 or 20, wherein L² is:

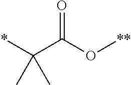

23. The nucleotide analog of claim 19 or 20, wherein L² is:

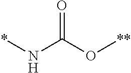

24. The nucleotide analog of any one of claims 19-23, wherein L² is attached to the base of the nucleotide.

25. A method of sequencing a single-stranded polynucleotide, comprising
a. incorporating the nucleotide analog of any one of claims 1-24 into a primer hybridized to said single-stranded polynucleotide using a polymerase;
b. detecting the identity of said nucleotide analog; and
c. contacting said nucleic acid molecule with a carbamatase.

26. The method of claim 25, wherein said carbamatase reacts with said incorporated nucleotide analog to expose a 3′ OH group.

27. The method of claim 25, wherein said carbamatase reacts with said incorporated nucleotide analog to cleave a linker bound to said base.

28. The method of claim 25, wherein said incorporating is accomplished via a polymerase.

29. The method of claim 25, wherein said nucleotide analog comprises or is bound to a label.

30. The method of claim 25, wherein detecting the identity of said nucleotide analog comprises detecting said label.

31. A method of labeling a nucleic acid molecule, comprising
a. incorporating the nucleotide analog of any one of claims 1-24 into the nucleic acid molecule, wherein said nucleotide analog comprises or is bound to a label; and
b. contacting the nucleic acid molecule with an esterase or an carbamatase.

32. The method of claim 31, further comprising detecting the identity of said label.

33. The method of claim 31, wherein said carbamatase reacts with said incorporated nucleotide analog to expose a 3′ OH group.

34. The method of claim 31, wherein a hydrolases such as a carbamatase, esterase or carbamatase reacts with said incorporated nucleotide analog to cleave a linker bound to said base.

35. The method of claim 31, wherein said incorporating is accomplished via a polymerase.

36. The method of claim 31, further comprising detecting the identity of the label before contacting said nucleic acid molecule with said esterase or carbamatase.

37. A method of synthesizing a single-stranded polynucleotide, comprising:
binding the nucleotide analog of any one of claims 1-24 to the 3′ hydroxyl end of a polynucleotide.

38. The method of claim 37, wherein said binding of said nucleotide analog to said polynucleotide is catalyzed using a polymerase.

39. The method of claim 37 or 38, further comprising contacting said nucleotide analog bound to said single-stranded polynucleotide with a carbamatase, wherein said carbamatase reacts with said nucleotide analog to expose the 3′ OH group of said nucleotide analog.

40. The method of claim 39, further comprising repeating said binding and contacting with said carbamatase.

41. The method of claim 39, wherein said carbamatase reacts with said incorporated nucleotide analog to cleave a linker bound to said base.

42. The method of claim 38, wherein said polymerase is a template-independent polymerase.

43. The method of claim 37, wherein said single-stranded polynucleotide is immobilized on a solid support.

44. The method of claim 37, wherein said nucleotide analog comprises or is bound to a label, further comprising detecting the identity of said label.
